SEQUENCE LISTING (12 sequences)

SEQ ID NO: 1 - DNA, 22 bp, synthetic construct
  source 1..22: mol_type = other DNA
ccatgggtcatatggcttgttc

SEQ ID NO: 2 - DNA, 18 bp, synthetic construct
  source 1..18: mol_type = other DNA
ggtaccaccaccaccacc

SEQ ID NO: 3 - DNA, 15 bp, synthetic construct
  source 1..15: mol_type = other DNA
ggtaccaacagcgac

SEQ ID NO: 4 - DNA, 20 bp, synthetic construct
  source 1..20: mol_type = other DNA
ggtgaccacggatccttagc

SEQ ID NO: 5 - DNA, 15 bp, synthetic construct
  source 1..15: mol_type = other DNA
ggtggtggtggtacc

SEQ ID NO: 6 - DNA, 39 bp, synthetic construct
  source 1..39: mol_type = other DNA
catatggcgtgcagcagtagcccgagcaaacattgcggc

SEQ ID NO: 7 - DNA, 513 bp, synthetic construct
  source 1..513: mol_type = other DNA
gctcttggtcaggacatggtgtcaccagaggccaccaactcttcttcctcctccttctcctctccttccagcgcgggaaggcatgtgcggagctacaatcaccttcaaggagatgtccgctggagaaagctattctctttcaccaagtactttctcaagattgagaagaacgggaaggtcagcgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtccactcatga

SEQ ID NO: 8 - DNA, 13423 bp, synthetic construct
  source 1..13423: mol_type = other DNA
catggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgactcgtccgtcctgtagaacgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaagctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagactcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacactcccccgccgtcgttcaatgagaatggataagaggctcgtgggattgacgtgagggggcagggatggctatatttctgggagggagaccacaacggtttcccactagaaataattttgtttaactttaagaaggagatatacccatggcatatggcgtgcagcagtagcccgagcaaacattgcggcggtggtggtggtaccgctcttggtcaggacatggtgtcaccagaggccaccaactcttcttcctcctccttctcctctccttccagcgcgggaaggcatgtgcggagctacaatcaccttcaaggagatgtccgctggagaaagctattctctttcaccaagtactttctcaagattgagaagaacgggaaggtcagcgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtccactcatgaggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataagaaattcaattaaggaaataaattaaggaaatacaaaaaggggggtagtcatttgtatataactttgtatgacttttctcttctatttttttgtatttcctccctttccttttctatttgtatttttttatcattgcttccattgaattggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgac

SEQ ID NO: 9 - protein, 5 aa, synthetic construct
GGGGT

SEQ ID NO: 10 - protein, 12 aa, synthetic construct
MACSSSPSLHCG

SEQ ID NO: 11 - protein, 170 aa, synthetic construct
ALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 12 - protein, 190 aa, synthetic construct
MGHMACSSSPSKHCGGGGGTALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

(19) United States
(12) Patent Application Publication
(10) Pub. No.: US 2025/0179135 A1
(43) Pub. Date: Jun. 5, 2025

(54) KERATINOCYTE GROWTH FACTOR (KGF)-TRANSDERMAL PEPTIDE (TP) FUSION PROTEIN, AND PREPARATION AND APPLICATION THEREOF

(71) Applicant: WENZHOU MEDICAL UNIVERSITY, Wenzhou (CN)

(72) Inventors: Xiaokun LI, Wenzhou (CN); Li LIN, Wenzhou (CN); Shuang GAO, Wenzhou (CN); Fanghua GONG, Wenzhou (CN); Lishang DAI, Wenzhou (CN); Zhenlin HU, Wenzhou (CN); Haitao XI, Wenzhou (CN); Jisheng MA, Wenzhou (CN); Yunpeng WANG, Wenzhou (CN); Nuo XU, Wenzhou (CN)

(21) Appl. No.: 19/027,921

(22) Filed: Jan. 17, 2025

Related U.S. Application Data
(63) Continuation of application No. PCT/CN2024/118439, filed on Sep. 12, 2024.

(30) Foreign Application Priority Data
Dec. 8, 2023 (CN) 202311679527.7

(51) Int. Cl. C07K 14/475; A61K 38/00; A61P 17/14; C07K 14/00; C12N 15/82; C12Q 1/6827; C12Q 1/686

(52) U.S. Cl. CPC C07K 14/475; A61P 17/14; C07K 14/001; C12N 15/8205; C12Q 1/6827; C12Q 1/686; A61K 38/00; C07K 2319/00

(57) ABSTRACT

A keratinocyte growth factor (KGF)-transdermal peptide (TP) fusion protein is provided. The amino acid sequence of the KGF-TP fusion protein is shown as SEQ ID NO: 12. The nucleotide sequence of a gene encoding the KGF-TP fusion protein is shown as SEQ ID NO: 8. A recombinant pGM3301 vector containing the gene is also provided. A preparation method and application of the KGF-TP fusion protein are also provided.

### REFERENCE TO AN ELECTRONIC SEQUENCE LISTING

The contents of the electronic sequence listing (Name: SequenceListing.xml; Size: 25,700 bytes; and Date of Creation: Nov. 26, 2024) is herein incorporated by reference in its entirety.

### CROSS-REFERENCE TO RELATED APPLICATIONS

This application is a continuation of International Patent Application No. PCT/CN2024/118439, filed on Sep. 12, 2024, which claims the benefit of priority from Chinese Patent Application No. 202311679527.7, filed on Dec. 8, 2023. The content of the aforementioned applications, including any intervening amendments thereto, are incorporated herein by reference.

### TECHNICAL FIELD

This application relates to fusion protein, and more particularly to a keratinocyte growth factor (KGF)-transdermal peptide (TP) fusion protein, and a preparation and application thereof.

### BACKGROUND

Keratinocyte growth factor-2 (KGF-2) is an important member of the family of fibroblast growth factors (FGFs). Yamasaki et al. (Yamasaki M, Miyake A, Tagashira S, Itoh N. Structure and expression of the rat mRNA encoding a novel member of the fibroblast growth factor family. J Biol Chem. 1996 Jul. 5; 271 (27): 15918-21) isolated a new cytokine from rat embryonic keratinocytes in 1996, which was named KGF-2. KGF-2 is a protein molecule composed of 208 amino acid residues with a signal peptide (40 residues) at the N-terminal. KGF-2 can promote the repair of epidermal and corneal damage, and participate in the whole process of lung development in the embryo. In addition, KGF-2 plays a significant role in promoting the hair growth. As a protein molecule, KGF-2 has lower side effects than minoxidil, and is considered as the most promising candidate in the development of drugs for promoting the hair growth.

Enhancing the stability and transdermal efficiency of transdermal drugs plays a crucial role in the research and development of hair growth-promoting agents. Therefore, in order to develop a hair growth-promoting agent derived from KGF-2, it is urgently required to find a way to improve the stability and transdermal rate of KGF-2. Some methods have been adopted in the prior art to improve the permeability of KGF-2, such as physical methods, in which the KGF is adsorbed on the microneedle and delivered directly to the dermis by the microneedle; and chemical methods, in which the KGF-2 is fused with polyethylene glycol (PEG) to increase the affinity to the cell membrane and delivered into the dermis. However, the above methods have an irreversible effect on the stability of KGF-2, and may also affect the user's experience.

### SUMMARY

An object of the disclosure is to provide a keratinocyte growth factor (KGF)-transdermal peptide (TP) fusion protein, and a preparation and application thereof, so as to solve the problems in the prior art that it has irreversible effects on the stability of KGF-2, and affects the user's experience.

In order to achieve the above object, the following technical solutions are adopted.

In a first aspect, this application provides a KGF-TP fusion protein, wherein an amino acid sequence of the KGF-TP fusion protein consists of SEQ ID NO: 12.

In a second aspect, this application provides a gene encoding the above KGF-TP fusion protein, wherein a nucleotide sequence of the gene consists of SEQ ID NO: 8.

In a third aspect, this application provides a recombinant vector, comprising the above gene.

In some embodiments, the recombinant vector is a pGM3301 vector.

In a fourth aspect, this application provides a recombinant strain, comprising the above recombinant vector.

In a fifth aspect, this application provides a hair growth-promoting agent, comprising the above KGF-TP fusion protein.

In a sixth aspect, this application provides a method for promoting hair growth in a subject in need thereof, comprising:
administering the above KGF-TP fusion protein to the subject.

In a seventh aspect, this application provides a method for preparing an Arabidopsis thaliana expression system capable of expressing the above KGF-TP fusion protein, comprising:
(1) ligating a TP-1 gene and a KGF-2 gene into a pGM3301 vector using a T4 ligase to obtain a ligation product; transforming the ligation product into Escherichia coli cells, and screening a target Escherichia coli cell comprising a gene encoding the KGF-TP fusion protein; and extracting a plasmid from the target Escherichia coli cell;
wherein a nucleotide sequence of the TP-1 gene consists of SEQ ID NO: 6, a nucleotide sequence of the KGF-2 gene consists of SEQ ID NO: 7, and a nucleotide sequence of the gene encoding the KGF-TP fusion protein consists of SEQ ID NO: 8;
(2) transforming the plasmid into EHA105 Agrobacterium competent cells by using a freeze-thaw method, and screening a target Agrobacterium cell comprising the plasmid by using a polymerase chain reaction (PCR) method followed by culture at 28° C. and 230 rpm to an absorbance OD₅₉₀ of 1.0 and centrifugation to collect the target Agrobacterium cell;
wherein the PCR method adopts a first pair of primers and a second pair of primers; a nucleotide sequence of a forward primer in the first pair of primers consists of SEQ ID NO: 1, and a nucleotide sequence of a reverse primer in the first pair of primers consists of SEQ ID NO: 2; and a nucleotide sequence of a forward primer in the second pair of primers consists of SEQ ID NO: 3, and a nucleotide sequence of a reverse primer in the second pair of primers consists of SEQ ID NO: 4; and
(3) mixing the target Agrobacterium cell with a transformation solution; immersing an inflorescence of an Arabidopsis thaliana plant into the transformation solution for transformation, followed by collection of a seed pod, drying in shade and hulling; continuously performing sowing for 2 generations to screen a homozygous transformed plant capable of expressing the KGF-TP fusion protein as the Arabidopsis thaliana expression system.

In an eighth aspect, this application provides a method for screening a transformed Arabidopsis thaliana plant capable of expressing the above KGF-TP fusion protein, comprising:
(1) extracting a whole genome of transformed Arabidopsis thaliana plants using a genome extraction kit, amplifying the whole genome by using a PCR method to obtain an amplified product, separating the amplified product through an agarose gel followed by detection using an ultraviolet (UV) imager;
wherein the PCR method adopts a first pair of primers and a second pair of primers; a nucleotide sequence of a forward primer in the first pair of primers consists of SEQ ID NO: 1, and a nucleotide sequence of a reverse primer in the first pair of primers consists of SEQ ID NO: 2; and a nucleotide sequence of a forward primer in the second pair of primers consists of SEQ ID NO: 3, and a nucleotide sequence of a reverse primer in the second pair of primers consists of SEQ ID NO: 4;
(2) pulverizing a tissue of an Arabidopsis thaliana plant which is identified by PCR to be successfully transformed, adding a tissue powder into a protein extraction buffer to obtain a first mixed solution, placing the first mixed solution in an ice bath followed by centrifugation at 4° C. to collect a supernatant, adding a loading buffer into the supernatant to obtain a second mixed solution, transferring the second mixed solution to a boiling water bath followed by sodium dodecyl sulfate-polyacrylamide gel electrophoresis (SDS-PAGE) to separate an Arabidopsis total soluble protein and transferring to a polyvinylidene fluoride (PVDF) membrane using a protein transfer apparatus; blocking the PVDF membrane at room temperature with a blocking buffer followed by washing, addition of an anti-KGF-2 mouse antibody and incubation at 4° C. overnight; washing the PVDF membrane, followed by addition of an alkaline phosphatase-labeled goat anti-mouse antibody and incubation at room temperature; washing the PVDF membrane followed by addition of 5-bromo-4-chloro-3-indolyl-phosphate/nitro blue tetrazolium (BCIP/NBT) as a color developing agent and detection using an imager.

Compared to the prior art, the present disclosure has the following beneficial effects.

(1) The Arabidopsis thaliana plant used in the present disclosure has no potentially pathogenic heat sources and endotoxins, indicating that it is a highly safe expression system. Moreover, the Arabidopsis thaliana is a eukaryotic organism with a complete protein post-translational modification function, ensuring that the expressed exogenous protein is soluble and biologically effective. In addition, the planting, cultivation and transformation methods of Arabidopsis thaliana are simple, which can significantly reduce the production cost of high value-added proteins. Therefore, the Arabidopsis-mediated production of KGF-TP fusion protein has obvious advantages and a brilliant application prospect.

(2) The present disclosure can improve the biological safety and stability of KGF-2 without affecting its biological activity. The fusion with TP-1-can significantly increase the transdermal rate of KGF-2, thereby greatly improving its application prospect in the hair growth promotion. At the same time, biological transdermal drug delivery can also reduce the pain caused by microneedles. Such a fusion protein production system and gel preparation have no potential risks and reduce skin damage.

(3) The fusion protein production system of the present disclosure is simple to prepare, and has obvious transdermal effect. According to the results of animal experiments, the fusion expression of TP-1 and KGF-2 has no influence on their respective structures and no adverse impact on test animals. Therefore, the fusion protein is a biosafe substance.

### BRIEF DESCRIPTION OF THE DRAWINGS

FIG. 1 is a partial schematic diagram of pGM3301-TP1-KGF2 in accordance with an embodiment of the present disclosure;

FIGS. 2A-D show an Agrobacterium-mediated transformation process of Arabidopsis thaliana in accordance with an embodiment of the present disclosure;

FIGS. 3A-B schematically illustrate polymerase chain reaction (PCR) detection of a TP1-KGF2 Arabidopsis thaliana plant in accordance with an embodiment of the present disclosure;

FIG. 4 is a Western-blot image of the TP1-KGF2 Arabidopsis thaliana plant in accordance with an embodiment of the present disclosure;

FIGS. 5A-B show hair growth-promoting effect of TP1-KGF2 in accordance with an embodiment of the present disclosure; and

FIGS. 6A-B show transdermal release ability of the TP1-KGF2 (green represents the detected fluorescence) in accordance with an embodiment of the present disclosure.

### DETAILED DESCRIPTION OF EMBODIMENTS

The technical solutions of the present disclosure will be clearly and completely described below in conjunction with the accompanying drawings and embodiments. Obviously, described below are only some embodiments of the present disclosure, instead of all embodiments of the present disclosure. Based on the embodiments in the present disclosure, all other embodiments obtained by those of ordinary skill in the art without making creative efforts shall fall within the scope of the present disclosure.

### EXAMPLE 1

Provided herein was a method for preparing an expression system of a keratinocyte growth factor (KGF)-transdermal peptide (TP) fusion protein.

### (1) Construction of TP1-KGF2 Fusion Gene Expression Vector

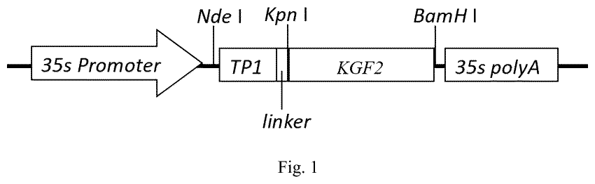

A TP-1 gene (the nucleotide sequence shown as SEQ ID NO: 6) on a cloning vector (the empty vector and Escherichia coli for cloning were purchased from TransGen Biotech Co. LTD) was cleaved using Nde I and Kpn I endonucleases. A KGF-2 gene (with the nucleotide sequence from GenBank, AB002097.1, shown as the FGF10 mRNA nucleic acid sequence included in SEQ ID NO: 7, and was optimized according to the codon preference of an Arabidopsis thaliana plant) stored in a cloning vector was cleaved using Kpn I and BamH I endonucleases. A pGM3301 vector was digested by Nde I and BamH I endonucleases. The obtained TP-1 gene fragment, KGF-2 gene fragment and the digested pGM3301 vector were recovered using a deoxyribonucleic acid (DNA) recovery kit, mixed in a molar ratio of 1:1:1, and ligated using a T4 ligase to obtain a ligation product. The ligation product was transformed into Escherichia coli cells. A target Escherichia coli cell containing the desired vector was screened. The desired vector was named pGM3301-TP1-KGF2 (with the nucleotide sequence shown as SEQ ID NO: 8; the linker is a part of the “TP1-KGF2” fusion protein, with the nucleotide sequence shown as SEQ ID NO: 5, which was flexible, had small steric hindrance, and served to link two proteins without affecting the properties of the fusion protein), as shown in FIG. 1, which was a partial schematic diagram of pGM3301-TP1-KGF2 of the present disclosure.

### (2) Agrobacterium-Mediated Transformation of Arabidopsis thaliana Plant

A pGM3301-TP1-KGF2 plasmid (with the amino acid sequence shown as SEQ ID NO: 12) was extracted as follows.

Step (1) 1-4 mL of a bacterial solution (pGM3301-TP1-KGF2) cultured overnight in a LB medium was extracted and centrifuged at 12,000×g for 1 min, and the supernatant was discarded.

Step (2) 250 μL of a Buffer S1 was added to the bacterial precipitate to obtain a suspension. The suspension should be uniform, and no small bacterial lumps should be left.

Step (3) The suspension was added with 250 μL of a Buffer S2, and gently and thoroughly inverted 4-6 times for mixing such that the bacteria were fully lysed until a transparent solution was formed. This step should take no more than 5 min.

Step (4) The transparent solution was added with 350 μL of a Buffer S3, gently and thoroughly inverted 6-8 times for mixing, and centrifuged at 12,000×g for 10 min to obtain a supernatant.

Step (5) The supernatant in step (4) was aspirated, transferred to a preparation tube, and centrifuged at 12,000×g for 1 min. The filtrate was discarded.

Step (6) The preparation tube was placed in a centrifuge tube, added with 500 μL of a Buffer W1, and centrifuged at 12,000×g for 1 min. The filtrate was discarded.

Step (7) The preparation tube was placed in the centrifuge tube, added with 700 μL of a Buffer W2, and centrifuged at 12,000×g for 1 min to collect the filter residue. The filtrate was discarded. The filter residue was washed once again with 700 μL of the Buffer W2 in the same way, and the filtrate was discarded.

Step (8) The preparation tube was placed in a 2 mL centrifuge tube followed by centrifugation at 12,000×g for 1 min.

Step (9) The preparation tube was transferred to a 1.5 mL centrifuge tube. 150 μL of an Eluent A was added to a center of the preparation tube membrane followed by standing at room temperature for 1 min and centrifugation at 12,000 ×g for 1 min, so as to obtain the pGM3301-TP1-KGF2 plasmid.

The specific components of each substance were as follows: Eluent A: deionized water; Buffer W2: ethanol and tris (hydroxymethyl) aminomethane hydrochloride (Tris-HCl); Buffer W1: isopropanol and Tris-HCl; Buffer S3: ammonium acetate (NH₄Ac); Buffer S2: NaOH and sodium dodecyl sulfate (SDS); Buffer S1: Tris-HCl, ethylenediaminetetraacetic acid (EDTA) and glucose, pH 8.0; and LB: sodium chloride, tryptone, yeast powder and sucrose, pH 7.0.

The pGM3301-TP1-KGF2 plasmid was transformed into EHA105 Agrobacterium competent cells by using a freeze-thaw method. A target Agrobacterium cell containing the pGM3301-TP1-KGF2 plasmid was identified and screened by using a polymerase chain reaction (PCR) method (the nucleotide sequence of a TP1 forward primer consists of SEQ ID NO: 1, and the nucleotide sequence of a TP1 reverse primer consists of SEQ ID NO: 2; and the nucleotide sequence of a KGF forward primer consists of SEQ ID NO: 3, and the nucleotide sequence of a KGF reverse primer consists of SEQ ID NO: 4), placed in a 2 L culture bottle in a shaker and cultured at 28° C. and 230 rpm for 20 h until an absorbance OD₅₉₀ of the Agrobacterium liquid reached 1.0. A target Agrobacterium cell was collected by centrifugation and mixed with 2 L of a transformation solution (4.3 g of Murashige&Skoog basal salt mixture (MS), 100 g of sucrose, 2 mL of a B-vitamin solution, 400 μL of Slilwet® L-77, 180 μL of 1M NaOH, and 1980 mL of double-distilled water (ddH₂O)). An inflorescence of the Arabidopsis thaliana plant was immersed into a transformation solution for 7 min for transformation. The Arabidopsis thaliana plant was transformed again in the same way at an interval of 1 day (the transformation solution was the same as the previous time). The transformed Arabidopsis thaliana plant was placed in the dark for 3 days. Seed pods of the transformed Arabidopsis thaliana plant were collected successively, dried in shade and hulled to collect seeds. The seeds were sown to obtain a transformed plant named T1, and the sowing was continuously performed for 2 generations to screen a homozygous transformed plant named T3 (genetic traits were basically stable after continuous sowing and screening for 3 generations).

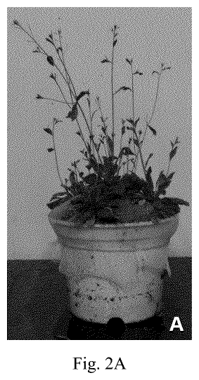

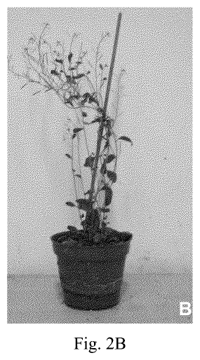

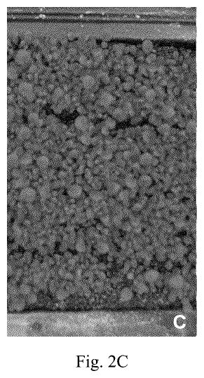

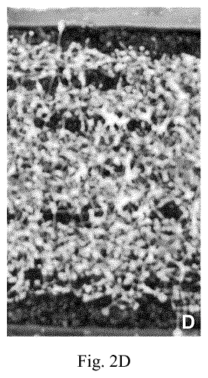

FIGS. 2A-D showed the Agrobacterium-mediated transformation process of Arabidopsis thaliana provided herein, where FIG. 2A referred to the bolting of wild Arabidopsis thaliana plants; FIG. 2B referred to the transformation of the wild Arabidopsis thaliana plants; FIG. 2C referred to the sowing of the transformed Arabidopsis thaliana plants; and FIG. 2D referred to the screening of the transformed Arabidopsis thaliana plants. As shown in FIGS. 2A-D, the resistant transformed plants were screened by applying a 1 wt. % glufosinate ammonium solution as a screening agent. In FIG. 2D, green represented the successfully transformed resistant Arabidopsis thaliana seedlings, and yellow represented the untransformed Arabidopsis thaliana seedlings.

### (3) Screening of Arabidopsis thaliana Plant Containing TP1-KGF2 Fusion Protein

A whole genome of transformed Arabidopsis thaliana plants T3 was extracted using a genome extraction kit (the nucleotide sequence of a TP1 forward primer consists of SEQ ID NO: 1, and the nucleotide sequence of a TP1 reverse primer consists of SEQ ID NO: 2; and the nucleotide sequence of a KGF forward primer consists of SEQ ID NO: 3, and the nucleotide sequence of a KGF reverse primer consists of SEQ ID NO: 4). The whole genome was amplified by running a program in a PCR instrument: 95° C., 5 min; (95° C., 30 s; 60° C., 30 s; 72° C., 70 s)×30 cycles; and 72° C., 8 min, so as to obtain an amplified product. The amplified product was separated through an agarose gel and detected using an ultraviolet (UV) imager.

A tissue of an Arabidopsis thaliana plant which is identified by PCR to be successfully transformed was pulverized to obtain a tissue powder. The tissue powder was added into a protein extraction buffer (a ratio of a mass of the tissue powder to a volume of the protein extraction buffer was 1 kg:2 L) to obtain a first mixed solution. The first mixed solution was placed in an ice bath for 2 h. 10,000 g of the first mixed solution was centrifuged at 10,000×g and 4° C. for 20 min to obtain a supernatant. The supernatant was added with a protein loading buffer to obtain a second mixed solution (a volume ratio of the first mixed solution to the protein loading buffer was 10:1). The second mixed solution was placed in a boiling water bath for 10 min. An Arabidopsis total soluble protein was separated through sodium dodecyl sulfate-polyacrylamide gel electrophoresis (SDS-PAGE), and transferred to a polyvinylidene fluoride (PVDF) membrane using a membrane transfer apparatus. The PVDF membrane was blocked with a blocking solution at room temperature for 2 h and washed to remove the blocking solution. The PVDF membrane was added with an anti-KGF-2 mouse antibody at dilution of 1:1000 and incubated at 4° C. overnight, and washed to remove the anti-KGF-2 mouse antibody. The PVDF membrane was added with an alkaline phosphatase-labeled goat anti-mouse antibody at dilution of 1:10000 and incubated at room temperature for 2 h, and washed to remove the alkaline phosphatase-labeled goat anti-mouse antibody. The PVDF membrane was added with 5-bromo-4-chloro-3-indolyl-phosphate/nitro blue tetrazolium (BCIP/NBT) as a color developing agent, and detected using an imager. In this way, three overexpressing transformed Arabidopsis thaliana plants, named NCF-365, NCF-669, and NCF-1470, were screened by molecular biological detection.

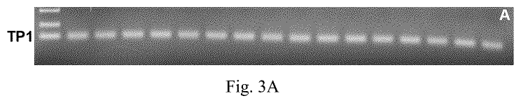

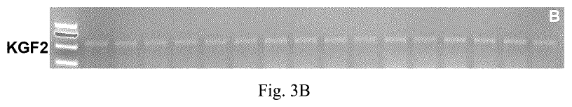

FIGS. 3A-B schematically illustrated PCR detection of the TP1-KGF2 Arabidopsis thaliana plant provided herein, where FIG. 3A referred to the PCR detection of the TP1 (about 100 bp) gene fragment; and FIG. 3B referred to the PCR detection of the KGF2 (about 500 bp) gene fragment. As shown in FIGS. 3A-B, the fusion gene TP1-KGF2 has been integrated into the genome of Arabidopsis thaliana.

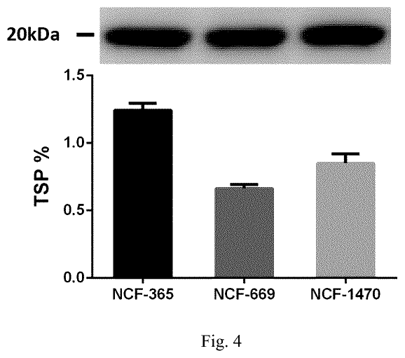

FIG. 4 showed a Western-blot image of the TP1-KGF2 Arabidopsis thaliana plant provided herein, where the horizontal axis represented NCF-365, NCF-669 and NCF-1470, and the vertical axis represented the total suspended particulate matter percentage (TSP %). As shown in FIG. 4, the fusion gene TP1-KGF2 can express the TP1-KGF2 protein in Arabidopsis thaliana.

### EXAMPLE 2 VERIFICATION OF HAIR GROWTH-PROMOTING ACTIVITY

### (1) Establishment of Alopecia Animal Model

A hair plucking method was adopted to induce alopecia to establish the alopecia animal model. Two groups of 7-week-old female C57BL/6 mice (4 mice in each group) were each anesthetized with a small animal gas inhalation anesthesia machine. Rosin and paraffin were mixed in a mass ratio of 1:1, heated for melting to obtain a depilatory wax. The depilatory wax was evenly applied to a back of each of the mice, and peeled off after solidifying. The above application, solidification and peeling operations were repeated 3 times until the back of each of the mice was smooth and hairless, so as to obtain two groups of alopecia mice.

### (2) Preparation of TP1-KGF2 Hair Regrowth Emulsion

2 g of palmitic acid, 0.875 mL of ethanol, 2.5 mL of an Arabidopsis thaliana extract (the Arabidopsis thaliana extract contained about 150 μg of TP1-KGF2 protein, and the components of Arabidopsis thaliana itself did not affect or synergize with hair growth promotion) and 50 mg of benzethonium chloride were vigorously stirred at 50° C. for mixing to obtain a uniform and stable cream. The Arabidopsis thaliana extract was added such that the resultant hair growth-promoting cream reagent contained about 1.0-10.0 mg/mL of the TP1-KGF2 protein.

### (3) Detection of Hair Growth-Promoting Effect of TP1-KGF

The first group of alopecia mice were treated as follows.

The cream was applied to the back of each of the first group of alopecia mice at an interval of 24 h, and the situation of hair growth was detected every 3 days, which served as an experimental group.

The second group of alopecia mice were treated as follows.

The second group of alopecia mice were treated without applying the cream, with the other conditions being the same as the first group, which served as a blank control group.

The two groups of mice were killed on day 15, and skin sample tissues of the mice were collected. The skin sample tissues were fixed on slices followed by cutting on a paraffin slicer to obtain tissue samples with a thickness of about 5-10 μm. The tissue samples were placed in a basin filled with tap water, and then soaked in 42° C. warm water for about 5-10 s to allow tissues to fully stretch. The tissue samples were taken out, naturally air-dried overnight, and then placed in a 60° C. oven for 2-4 h. The tissue samples were baked in an oven for 20 min before use, and added with xylene while hot for dewaxing. The specific steps were as follows. To-be-tested tissue samples were placed in xylene and fully soaked for 15 min. After soaking, xylene was replaced, and the to-be-tested tissue samples were soaked continuously for 15 min. The to-be-tested tissue samples were placed in anhydrous ethanol, soaked for 5 min, then taken out and soaked in anhydrous ethanol again for 5 min, such that the xylene used in the dewaxing process was washed off, allowing water to enter the to-be-tested tissue samples. The to-be-tested tissue samples were washed with 100%, 90%, 80%, and 70% ethanol in sequence. Each of the to-be-tested tissue samples was added with 100 μL of a pre-prepared hematoxylin staining solution by a pipette for 7-10 min for fully staining. After staining, the excess hematoxylin staining solution was washed off with distilled water. Then, the to-be-tested tissue samples were differentiated with a differentiation solution for 30 s, rinsed with ddH₂O, and added with eosin dye for 3 min for fully staining. After staining, the to-be-tested tissue samples were subjected to gradient dehydration. The specific steps of the gradient dehydration were performed with: 80% ethanol dehydration for 5 s, 100% ethanol dehydration for 2 min and anhydrous ethanol dehydration for 2 min. The to-be-tested tissue samples were soaked in xylene twice for 15 min each time followed by sucking away the xylene and sealing with a neutral gum.

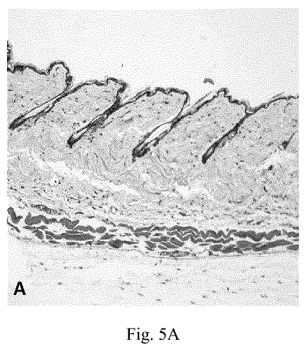

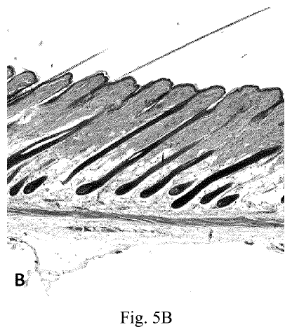

FIGS. 5A-B showed hair growth-promoting effect of TP1-KGF provided herein, where FIG. 5A referred to a hematoxylin and eosin (H&E)-stained tissue sample of the mouse not given TP1-KGF2; and FIG. 5B referred to a H&E-stained tissue sample of the mouse given TP1-KGF2. It can be seen from FIGS. 5A-B that, compared with the control group, hair follicles of the mice given TP1-KGF2 in the experimental group regrew hair.

### (4) Detection of Transdermal Release Ability of TP1-KGF2

The tissue samples sealed with the neutral gum were heated under 60-80 kPa, taken out for cooling, washed with phosphate-buffered saline (PBS), added with 30 μL of 5% bovine serum albumin (BSA) as a blocking solution and blocked for 1 h. The tissue samples were washed to remove the blocking solution, added with an anti-KGF2 antibody at dilution of 1:250 and incubated at 4° C. overnight. The tissue samples were washed to remove the anti-KGF2 antibody, added with an TP1-KGF2 antibody at dilution of 1:500 and incubated at room temperature for 1 h. The tissue samples were dried, added with an appropriate amount of diaminobenzidine (DAB) as a color developing solution, and subjected to microscopic examination using a microscope. The staining proceeded until the tissue samples turned brownish-yellow. The steps of washing the tissue samples were as follows. The tissue samples were immersed in 70%, 80%, 90%, and 100% ethanol in sequence for dehydration, sealed with a neutral resin after completely dehydrated, and observed using a microscope.

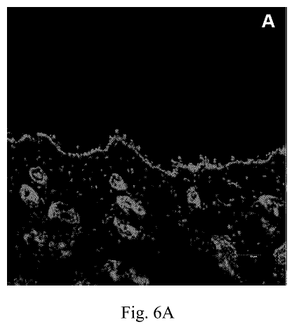

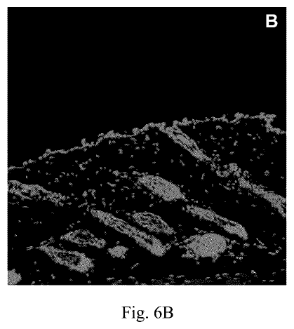

FIGS. 6A-B showed transdermal release ability of TP1-KGF2 provided herein (green represented the detected fluorescence), where FIG. 6A referred to the immunofluorescence detection of hair follicles given KGF-2; and FIG. 6B referred to the immunofluorescence detection of hair follicles given TP1-KGF2. As shown in FIGS. 6A-B, there was white detected fluorescence in FIG. 6B, indicating that TP1-KGF2 had penetrated the hair follicle epidermis; while there was no white detected fluorescence in FIG. 6A, indicating that KGF2 failed to entered the hair follicles or had been licked off by the mice mutually. Therefore, a clear fluorescence signal was detected after administration of TP1-KGF2, while it was difficult to detect a fluorescence signal after administration of KGF-2.

The embodiments described above are merely illustrative of the present application, and are not intended to limit the scope of the present application. For those of ordinary skill in the art, various modifications and replacements made without departing from the spirit and scope of the present disclosure shall fall within the scope of the present disclosure defined by the appended claims.

What is claimed is:

1. A keratinocyte growth factor (KGF)-transdermal peptide (TP) fusion protein, wherein an amino acid sequence of the KGF-TP fusion protein consists of SEQ ID NO: 12.

2. A gene encoding the KGF-TP fusion protein of claim 1, wherein a nucleotide sequence of the gene consists of SEQ ID NO: 8.

3. A recombinant vector, comprising:
the gene of claim 2.

4. The recombinant vector of claim 3, wherein the recombinant vector is a pGM3301 vector.

5. A recombinant strain, comprising:
the recombinant vector of claim 3.

6. A hair growth-promoting agent, comprising:
the KGF-TP fusion protein of claim 1.

7. A method for promoting hair growth in a subject in need thereof, comprising:
administering the KGF-TP fusion protein of claim 1 to the subject.

8. A method for preparing an Arabidopsis thaliana expression system capable of expressing the KGF-TP fusion protein of claim 1, comprising:
(1) ligating a TP-1 gene and a KGF-2 gene into a pGM3301 vector using a T4 ligase to obtain a ligation product; transforming the ligation product into Escherichia coli cells, and screening a target Escherichia coli cell comprising a gene encoding the KGF-TP fusion protein; and extracting a plasmid from the target Escherichia coli cell;
wherein a nucleotide sequence of the TP-1 gene consists of SEQ ID NO: 6, a nucleotide sequence of the KGF-2 gene consists of SEQ ID NO: 7, and a nucleotide sequence of the gene encoding the KGF-TP fusion protein consists of SEQ ID NO: 8;
(2) transforming the plasmid into EHA105 Agrobacterium competent cells by using a freeze-thaw method, and screening a target Agrobacterium cell comprising the plasmid by using a polymerase chain reaction (PCR) method followed by culture at 28° C. and 230 rpm to an absorbance OD₅₉₀ of 1.0 and centrifugation to collect the target Agrobacterium cell;
wherein the PCR method adopts a first pair of primers and a second pair of primers; a nucleotide sequence of a forward primer in the first pair of primers consists of SEQ ID NO: 1, and a nucleotide sequence of a reverse primer in the first pair of primers consists of SEQ ID NO: 2; and a nucleotide sequence of a forward primer in the second pair of primers consists of SEQ ID NO: 3, and a nucleotide sequence of a reverse primer in the second pair of primers consists of SEQ ID NO: 4; and
(3) mixing the target Agrobacterium cell with a transformation solution; immersing an inflorescence of an Arabidopsis thaliana plant into the transformation solution for transformation, followed by collection of a seed pod, drying in shade and hulling; continuously performing sowing for 2 generations to screen a homozygous transformed plant capable of expressing the KGF-TP fusion protein as the Arabidopsis thaliana expression system.

9. A method for screening a transformed Arabidopsis thaliana plant capable of expressing the KGF-TP fusion protein of claim 1, comprising:
(1) extracting a whole genome of transformed Arabidopsis thaliana plants using a genome extraction kit, amplifying the whole genome by using a PCR method to obtain an amplified product, separating the amplified product through an agarose gel followed by detection using an ultraviolet (UV) imager;
wherein the PCR method adopts a first pair of primers and a second pair of primers; a nucleotide sequence of a forward primer in the first pair of primers consists of SEQ ID NO: 1, and a nucleotide sequence of a reverse primer in the first pair of primers consists of SEQ ID NO: 2; and a nucleotide sequence of a forward primer in the second pair of primers consists of SEQ ID NO: 3, and a nucleotide sequence of a reverse primer in the second pair of primers consists of SEQ ID NO: 4; and
(2) pulverizing a tissue of an Arabidopsis thaliana plant which is identified by PCR to be successfully transformed, adding a tissue powder into a protein extraction buffer to obtain a first mixed solution, placing the first mixed solution in an ice bath followed by centrifugation at 4° C. to collect a supernatant, adding a loading buffer into the supernatant to obtain a second mixed solution, transferring the second mixed solution to a boiling water bath followed by sodium dodecyl sulfate-polyacrylamide gel electrophoresis (SDS-PAGE) to separate an Arabidopsis total soluble protein and transferring to a polyvinylidene fluoride (PVDF) membrane using a protein transfer apparatus; blocking the PVDF membrane at room temperature with a blocking buffer followed by washing, addition of an anti-KGF-2 mouse antibody and incubation at 4° C. overnight; washing the PVDF membrane, followed by addition of an alkaline phosphatase-labeled goat anti-mouse antibody and incubation at room temperature; washing the PVDF membrane followed by addition of 5-bromo-4-chloro-3-indolyl-phosphate/nitro blue tetrazolium (BCIP/NBT) as a color developing agent and detection using an imager.
